(19) United States
(12) Patent Application Publication
(10) Pub. No.: US 2025/0135617 A1
(43) Pub. Date: May 1, 2025

(54) Differential density system for mechanical devices

(71) Applicant: Ben Ciechanover, Edmonton (CA)

(72) Inventor: Ben Ciechanover, Edmonton (CA)

(21) Appl. No.: 18/499,221

(22) Filed: Nov. 1, 2023

(51) Int. Cl. B25D 1/08; A61B 9/00; B25D 1/14

(52) U.S. Cl. CPC B25D 1/08; A61B 9/00; B25D 1/14

(57) ABSTRACT

A system composed of different densities of materials making up a device for the assessment, treatment, diagnosis, advertisement, modelling, or otherwise impacting another object, person, living thing, or material. A method for distributing the centre of mass of a device by placing dense material or materials distal to the handle thereby managing the mechanical advantage of the instrument. The dense material is positioned within the shaft of the instrument, thereby allowing the distal head to be made partially or entirely out of an elastic polymer, organic, or inorganic substance. Modulation of the characteristics of the device allows for modulating of the impulse or striking force elicited by the device. The device may therefore be swung along an axis with resultant torque elicited. This device may be used for medical, industrial, personal, or other purposes.

### FIELD OF THE INVENTION

The present invention provides a device, tool, or accessory used for elicitation of torque and mechanical advantage along an axis before impacting another object, person, or material with a predictable impulse, percussion, impact force. Among the uses for the invention is the elicitation of deep tendon responses on neurological exams by delivering a consistent and predictable impulse, impact force, or percussion, thereby enabling more reliable assessments while limiting pain via excessively forceful impact. Other applications include percussion of kegs of alcoholic beverages, fruit, walls under construction, and other.

### BACKGROUND

Swinging devices along an axis is arguably among the most ancient of human tools. This includes hammers, axes, and countless other hand-held devices. In recent years the amplification of force via centripetal force has also been used in industrial and automatic applications and is expected to expand. Increasing the mechanical advantage increases the force applied. However, while the impact force or impulse is generated, excess in the amplitude of the force may lead to detrimental and uncontrolled effects, and therefore benefits from modulation. This modulation may be provided by adjusting or limiting the length of the shaft of the device, utilizing denser and or heavier material towards the distal end of the device in reference to the axis of use, or utilizing an elastic material used for striking of the target. Simultaneously, utilizing an excessively powerful percussion of an object may result in damage, breakage, or injury to the target being stuck. Therefore, modulating the forces listed above enables the user or application to provide a safer and more effective delivery of the force.

In addition, freeing the head of the device to be made up of polymer by maintaining a dense weighed portion among or within the shaft of the device allows for greater flexibility in the shapes of the head and directionality of the impact.

### SUMMARY

The invention provides for a method to construct devices, tools, instruments, or accessories used in the elicitation of impulse, percussion, or impact force by utilizing differences in the density the materials to modulate the force applied onto the target being struck against. The invention outlines multiple ways to modulate this force, which includes but is not limited to the length of the shaft, weight of the device, mechanical advantage along the axis of the device, and elasticity of the head of the device. Placing a higher density within or among the distal aspect of the shaft of the device increases its mechanical advantage while freeing the head of the device to be made of a lower density material such as a polymer, which further modulates the force applied while increasing safety and effectiveness of the device.

### DESCRIPTION OF THE FIGURES

FIG. 1 is a diagram of 5 types of mechanical devices utilizing differences in density. The white color represents low density of material or a hollow shaft. The grey color represents medium density, such as that of a polymer. The black color represents high density of material.

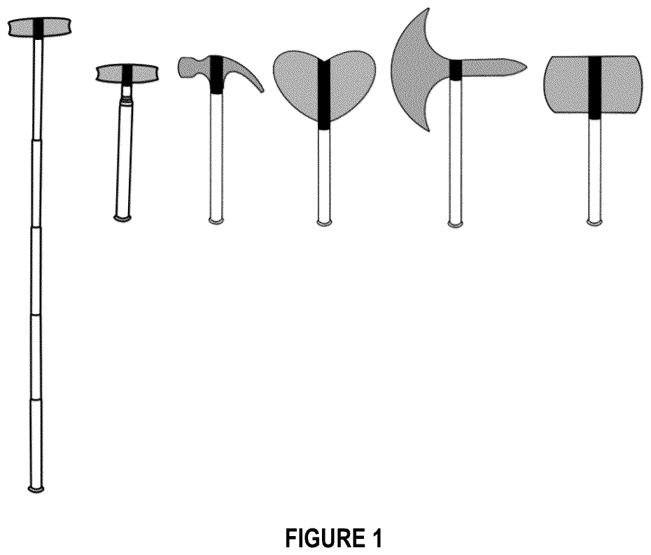

FIG. 2 is a diagram showing a reflex hammer with a denser weighted portion indicated in black, elastic polymer in grey, hollow telescopic shaft that is collapsed on the left and expanded on the right. The white color represents low density of material or a hollow shaft. The proximal end of the reflex hammer contains an elevated edge that may be used for the elicitation of deep pain responses.

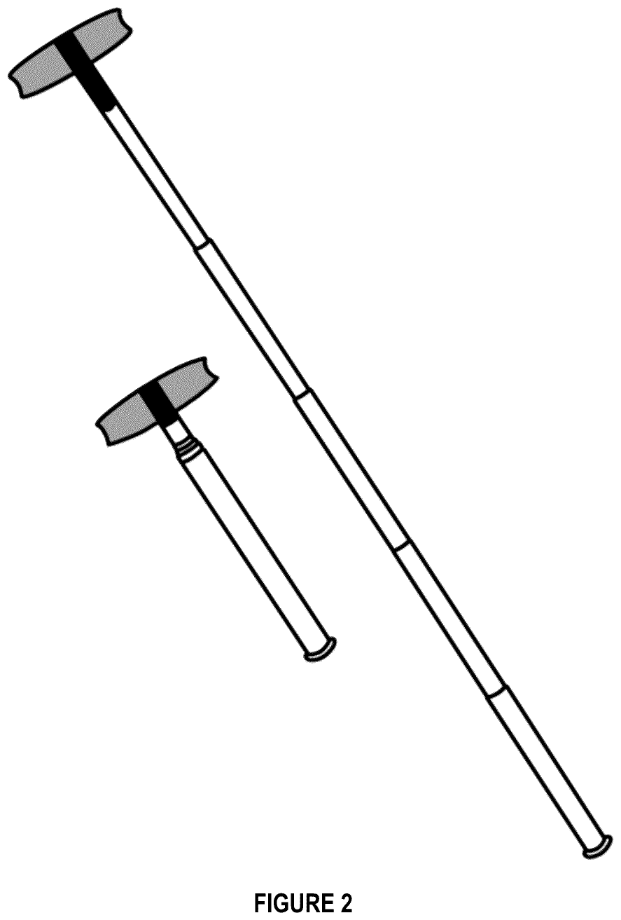

FIG. 3. Is a diagram of a hammer with a denser weighted portion indicated in black, elastic polymer in grey, and low density or hollow shaft indicated in white color. Using specific parameters this example shows how a hammer may be constructed with an elastic polymer head and used as a reflex hammer.

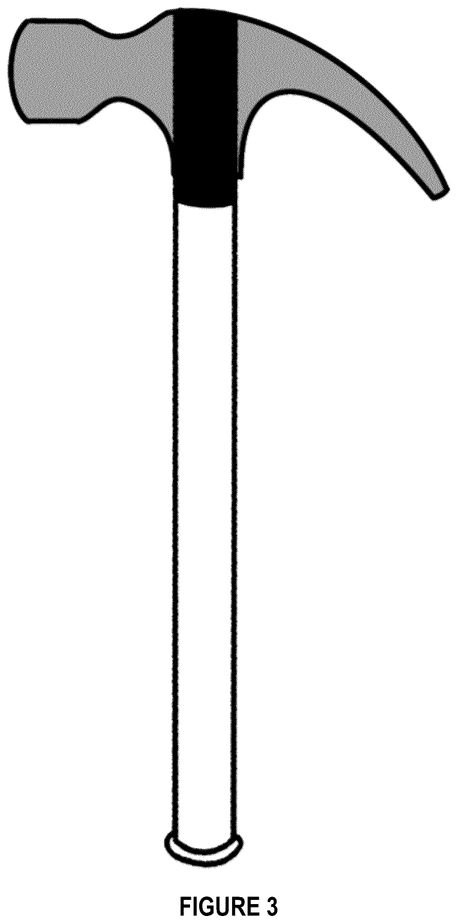

FIG. 4. Is a heart shaped reflex hammer with a denser weighted portion indicated in black, elastic polymer in grey, and low density or hollow shaft indicated in white color. Using specific parameters this example shows how a heart shape may be constructed with an elastic polymer head and used as a reflex hammer.

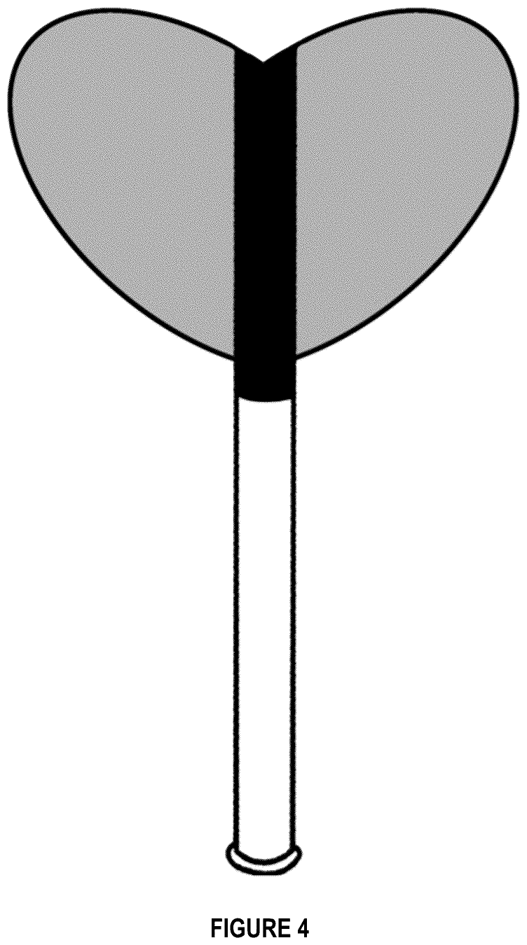

FIG. 5. Is a tomahawk or axe with a denser weighted portion indicated in black, elastic polymer in grey, and low density or hollow shaft indicated in white color. Using specific parameters this example shows how a tomahawk or axe shape may be constructed with an elastic polymer head and used as a reflex hammer.

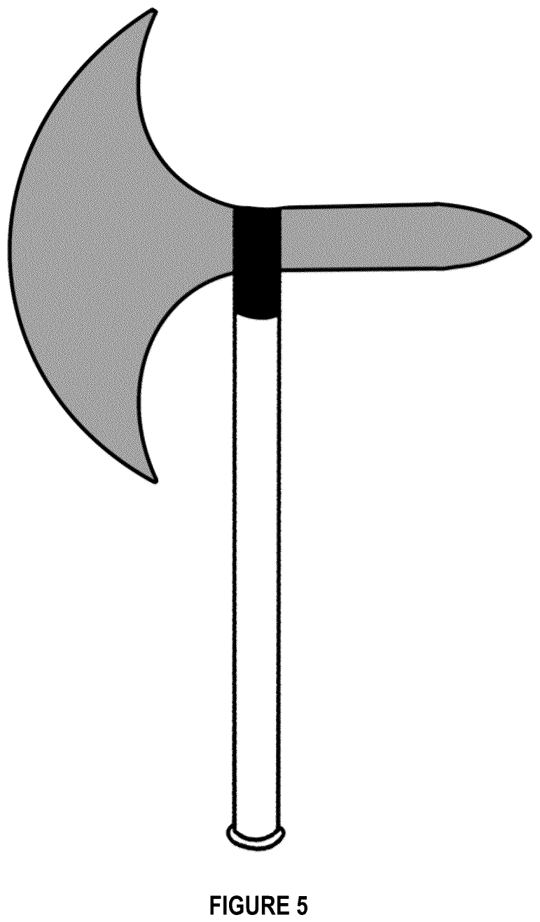

FIG. 6. Is a rubber mallet with a denser weighted portion indicated in black, elastic polymer in grey, and low density or hollow shaft indicated in white color. Using specific parameters this example shows how a safer and more effective rubber mallet may be constructed.

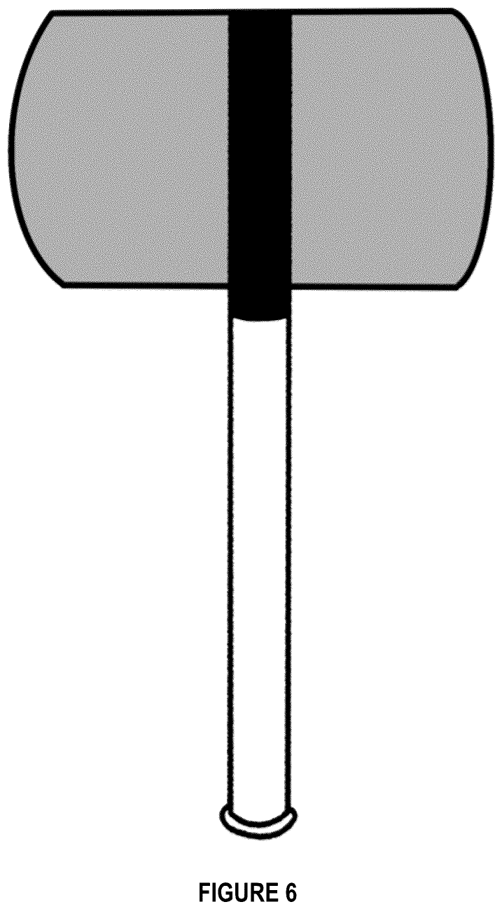

### DETAILED DESCRIPTION

Embodiments are described more fully below in sufficient detail to enable those skilled in the art to practice the system and method. However, embodiments may be implemented in many different forms and should not be construed as being limited to the embodiments set forth herein. The following detailed description is, therefore, not to be taken in a limiting sense.

The present invention provides the description and specific examples of the differential density system within mechanical devices for the modulation of percussion, impulse, or impact force elicited by the device onto another object, person, living thing, or material.

As used herein, a “device” refers to an object made for a particular purpose, especially when used in a mechanical form.

As used herein, a “shaft” refers to a long, narrow part or section forming the body or handle of a device or tool.

As used herein, “percussion” refers to striking of an object with or against another with some degree of force.

As used herein, “density” refers to the degree of compactness of a substance or material, especially in the context of mass.

As used herein, “modulation” refers to the exertion of a modifying or controlling influence onto another object, person, or material.

As used herein, “mass” refers to the intrinsic property of matter involving the resistance that the body of mater offers to a change in its speed or position upon application of a force.

As used herein, “polymer” refers to a substance that has a molecular structure consisting of a large number of similar units bonded together.

As used herein, “elastic” refers to an object or material that is able to resume its normal shape spontaneously after contraction, dilation, or distortion.

As used herein, “head” refers to the distal part of an object, person, or animal, often separated from the rest of the body or object. Within the context of the device, “head” refers to any material constructed near the distal portion of the device that may be intended to strike or percuss against another item.

As used herein, “tool” refers to a device or implement, especially held in the hand yet may be used otherwise as well, used to carry out a particular function.

As used herein, “mechanical advantage” refers to the ratio of the force produced by a machine to the force applied to it.

### EXAMPLES

### Example 1

Reflex hammers are used is the elicitation of deep tendon reflex responses. Some of the sites traditionally used for deep tendon reflexes include the biceps tendon, brachioradialis tendon, triceps tendon, pectoralis tendon, patellar tendon, Achilles tendon, yet other reflex responses may be elicited in many other skeletal muscles as well. The deep tendon reflex is caused by the stretch action on the tendon elicited by an outside force, causing a muscle response by the contraction of the muscle triggered by the tendon stretch. However, delivering a consistent impulse against the tendon of a patient is sometimes not sufficient for a reliable tendon response as patients have a diversity of tendon lengths, fatty composition above and below the tendon, along with other individual differences. By enabling modulation of the impact force and limiting excessively high impulses to the patient's tendons we reduce pain and risk of injury to patients.

### Example 2

Woodworking hammers, also called rubber mallets, are used to percuss rigid yet potentially fragile materials if excessive impulse is applied, while requiring high force distributed over the surface of impact. Traditional hammers and mallets often feature a rubber mallet with a hollow head filled with lead shot that delivers a solid blow without damaging the work. However, filling the rubber mallet with lead while increasing the mechanical advantage of the strike also introduces a rigid object within the head of the hammer, thereby increasing the risk to the work. By incorporating a dense material within the shaft of the hammer, and simultaneously utilizing a full elastic mallet, woodworking hammers become more effective yet safer for their target as they incorporate greater area of deformation of the rubber or polymer.

Although the above embodiments have been described in language that is specific to certain structures, elements, compositions, and methodological steps, it is to be understood that the technology defined in the appended claims is not necessarily limited to the specific structures, elements, compositions and/or steps described. Rather, the specific aspects and steps are described as forms of implementing the claimed technology. Since many embodiments of the technology can be practiced without departing from the spirit and scope of the invention, the invention resides in the claims hereinafter appended.

1. A device, tool, instrument, or accessory constituting multiple densities of materials.

2. The device of claim 1, wherein the distal aspect of the device consists of higher density material within or made up of the shaft.

3. The device of claim 1, wherein the distal aspect of the device consists of an elastic head made of polymer, organic, or inorganic substance.

4. The device of claim 1, wherein the shaft of the device may be telescopic, expandible, or of constant length.

5. The device of claim 1, wherein the proximal aspect of the device may contain, house, feature, or otherwise display another device, tool, or other accessory not otherwise specified.

6. The device of claim 1, wherein the shaft may be made up of polymer, metal, organic, or inorganic substance.

7. The device of claim 1, wherein the higher density material is made up of a polymer, metal, organic, or inorganic substance or substances.

8. The device of claim 1, wherein the device may be hand-held, used by another machinery, robot, animal or other while a proximal end of the device is affixed manually, mechanically, or automatically.

9. The device of claim 1, wherein the shaft of the device may be of proportional length to the application in use.

10. The device of claim 1, wherein the mass of the device may be adjusted per the application in use.
